(19) United States
(12) Patent Application Publication
(10) Pub. No.: US 2014/0194523 A1
(43) Pub. Date: Jul. 10, 2014

(54) DESENSITIZING DRUG PRODUCTS AND METHODS OF USE

(71) Applicant: Absorption Pharmaceuticals, LLC, Las Vegas, NV (US)

(72) Inventors: Ronald Franklin Gilbert, Huntington Beach, CA (US); Jeff Abraham, Las Vegas, NV (US)

(73) Assignee: Absorption Pharmaceuticals, LLC, Las Vegas, NV (US)

(21) Appl. No.: 14/146,582

(22) Filed: Jan. 2, 2014

Related U.S. Application Data
(60) Provisional application No. 61/749,096, filed on Jan. 4, 2013.

(51) Int. Cl. A61K 31/167

(52) U.S. Cl. CPC A61K 31/167
USPC 514/626

(57) ABSTRACT

Desensitizing drug products, methods of making desensitizing drug products, and methods of using desensitizing drug products including delivery of desensitizing drug products are disclosed herein.

### CROSS-REFERENCE TO RELATED APPLICATION

This application claims the benefit of priority under 35 U.S.C §119(e) of U.S. Provisional Application No. 61/749,096 filed Jan. 4, 2013, entitled “DESENSITIZING DRUG PRODUCTS AND METHODS OF USE”, the disclosure of which is incorporated herein by reference in its entirety.

### FIELD

The field relates to desensitizing drug products, methods of making desensitizing drug products and methods of using desensitizing drug products, including delivery of desensitizing drug products.

### DESCRIPTION OF THE RELATED ART

Anesthetic products are known in the medical field and can be used for desensitization. EMLA® (lidocaine 2.5% and prilocaine 2.5%) cream has been used as a topical anesthetic. EMLA comprises lidocaine and prilocaine in an emulsified topical cream. Lidocaine is recognized as being safe and effective. It has been reported, however, that prilocaine use results in metabolites that are responsible for methemoglobinemia. Accordingly, alternatives to. EMLA have been sought. See U.S. Pat. No. 6,299,902.

### SUMMARY

Example embodiments described herein have several features, no single one of which is solely responsible for their desirable attributes. Without limiting the scope of the claims, some of the advantageous features will now be summarized.

Provided herein are formulations for the treatment of disorders or conditions including premature ejaculation, interstitial cystiti s, and/or vulvody nia. Such treatment may include alleviation of certain symptoms of the conditions and disorders. In some embodiments, the symptom alleviated is pain. Such compositions may also be used in dermatological applications on keratinized and/or non-keratinized skin. Such compositions include desensitizing drug products.

The compositions may be contained within, among other things, metered spray bottles labeled for the treatment of premature ejaculation in males, interstitial cystitis, and/or vulvodynia wherein the metered spray bottles comprise genital desensitizer compositions, wherein the genital desensitizer compositions comprise an anesthetic agent; a first melting point depressing agent selected from the group consisting of thymol, methyl salicylate, phenyl salicylate, butylated hydroxytoluene, butylated hydroxyanisole, S(+)-ibuprofen, R(−)-ibuprofen, cineole, eugenol, capsaicin, eucalyptol, and an alcohol; and a second melting point depressing agent selected from the group consisting of thymol, methyl salicylate, phenyl salicylate, butylated hydroxytoluene, butylated hydroxyanisole, S(+)-ibuprofen, R(−)-ibuprofen, cineole, eugenol, capsaicin, eucalyptol, and an alcohol, and wherein the second melting point depressing agent is different from the first melting point depressing agent. In some embodiments, the compositions do not contain alcohol, or only contain a negligible amount of alcohol.

Methods for treating premature ejaculation in males also are provided. The methods include the step of administering to a subject a genital desensitizer composition, wherein the genital desensitizer compositions comprise an anesthetic agent; a first melting point depressing agent selected from the group consisting of thymol, methyl salicylate, phenyl salicylate, butylated hydroxytoluene, butylated hydroxyanisole, S(+)ibuprofen, R(−)-ibuprofen, cineole, eugenol, capsaicin, eucalyptol, and an alcohol; and a second melting point depressing agent selected from the group consisting of thymol, methyl salicylate, phenyl salicylate, butylated hydroxytoluene, butylated hydroxyanisole, S(+)-ibuprofen, R(−)-ibuprofen, cineole, eugenol, capsaicin, eucalyptol, and an alcohol, and wherein the second melting point depressing agent is different from the first melting point depressing agent. In some embodiments, the compositions do not contain alcohol, or only contain a negligible amount of alcohol.

Methods for the treatment of interstitial cystitis also are provided. The methods include the step of administering to a subject a genital desensitizer composition, wherein the genital desensitizer compositions comprise an anesthetic agent; a first melting point depressing agent selected from the group consisting of thymol, methyl salicylate, phenyl salicylate, butylated hydroxytoluene, butylated hydroxyanisole, S(+)ibuprofen, R(−)-ibuprofen, cineole, eugenol, capsaicin, eucalyptol, and an alcohol; and a second melting point depressing agent selected from the group consisting of thymol, methyl salicylate, phenyl salicylate, butylated hydroxytoluene, butylated hydroxyanisole, S(+)-ibuprofen, R(−)-ibuprofen, cineole, eugenol, capsaicin, eucalyptol, and an alcohol, and wherein the second melting point depressing agent is different from the first melting point depressing agent. In some embodiments, the compositions do not contain alcohol, or only contain a negligible amount of alcohol.

Methods for the treatment of vulvodynia also are provided. The methods include the step of administering to a subject a genital desensitizer composition, wherein the genital desensitizer compositions comprise an anesthetic agent; a first melting point depressing agent selected from the group consisting of thymol, methyl salicylate, phenyl salicylate, butylated hydroxytoluene, butylated hydroxyanisole, S(+)ibuprofen, R(−)-ibuprofen, cineole, eugenol, capsaicin, eucalyptol, and an alcohol; and a second melting point depressing agent selected from the group consisting of thymol, methyl salicylate, phenyl salicylate, butylated hydroxytoluene, butylated hydroxyanisole, S(+)-ibuprofen, R(−)-ibuprofen, cineole, eugenol, capsaicin, eucalyptol, and an alcohol, and wherein the second melting point depressing agent is different from the first melting point depressing agent. In some embodiments, the compositions do not contain alcohol, or only contain a negligible amount of alcohol.

The administering can be performed using a metered spray. The administering can include 3 to 10 sprays (e.g., 3, 4, 5, 6, 7, 8, 9, or 10 sprays in one or a series of applications). Each spray can include about 10 mg lidocaine. In certain embodiments, each spray may result in about 130 mcL of product being expelled.

The anesthetic agent can be lidocaine. In certain embodiments, prilocaine is not used or is not present. In an embodiment, the anesthetic agent is lidocaine, the first melting point depressing agent is thymol, and the second melting point depressing agent is ethanol.

In various embodiments, compositions and methods are provided that are more effectively delivered to the user and thus safer than compositions and methods known in the art. For example, a eutectic compositions (e.g., comprising an anesthetic and a first melting point depressing agent, together with a second melting point depressing agent for forming an emulsion) may (i) facilitate/enhance delivery of an anesthetic (e.g., lidocaine) through the stratum corneum, directly to nerves in the dermis. The composition, in accordance with some embodiments, more safely delivers the anesthetic agent to desensitize the nerves in the patient by creating a positive charge on the anesthetic, which reduces absorption into the bloodstream (e.g., systemic absorption). In some embodiments, the smaller amount of anesthetic may be used in formulations.

In an embodiment, a method of treating interstitial cystitis in a patient includes the steps of: administering to the patient a pharmaceutical composition, the composition including 7.7% by weight lidocaine based on the total weight of the pharmaceutical composition, 0.86% thymol based on the total weight of the pharmaceutical composition, 10% by weight ethanol based on the total weight of the pharmaceutical composition, and a base, and relieving symptoms of interstitial cystitis. In some embodiments, the pharmaceutical composition does not include prilocaine. According to some embodiments, the base includes an aqueous solution, and the aqueous solution has a pH in the range of about 7.4 to about 8.9. In some embodiments, the aqueous solution comprises a pH of about 7.4. In other embodiments, the aqueous solution comprises a pH of about 7.9. In some embodiments, the aqueous solution comprises a pH of about 8.4. According to some embodiments, the administering is performed using a metered spray.

In an embodiment, a method of treating vulvodynia in a patient includes the steps of: administering to the patient a pharmaceutical composition, the composition including 7.7% by weight lidocaine based on the total weight of the pharmaceutical composition, 0.86% thymol based on the total weight of the pharmaceutical composition, 10% by weight ethanol based on the total weight of the pharmaceutical composition, and a base, and relieving symptoms of vulvodynia. In some embodiments, the pharmaceutical composition does not include prilocaine. According to some embodiments, the base includes an aqueous solution, and the aqueous solution has a pH in the range of about 7.4 to about 8.9. In some embodiments, the aqueous solution comprises a pH of about 7.4. In other embodiments, the aqueous solution comprises a pH of about 7.9. In some embodiments, the aqueous solution comprises a pH of about 8.4. According to some embodiments, the administering is performed using a metered spray.

According to some embodiments a pharmaceutical composition for the treatment of premature ejaculation includes: 7.7% by weight lidocaine based on the total weight of the pharmaceutical composition, 0.86% ethyl vanillin based on the total weight of the pharmaceutical composition, 10% by weight ethanol based on the total weight of the pharmaceutical composition, and a base. In some embodiments, the pharmaceutical composition does not include prilocaine. According to some embodiments, the composition comprises a viscosity sufficient to permit delivery of the composition through a spray bottle.

### DETAILED DESCRIPTION

Although certain preferred embodiments and examples are disclosed below, inventive subject matter extends beyond the specifically disclosed embodiments to other alternative embodiments and/or uses, and to modifications and equivalents thereof Thus, the scope of the disclosure is not limited by any of the particular embodiments described below. For example, in any method or process disclosed herein, the acts or operations of the method or process may be performed in any suitable sequence and are not necessarily limited to any particular disclosed sequence. For purposes of contrasting various embodiments with the prior art, certain aspects and advantages of these embodiments are described. Not necessarily all such aspects or advantages are achieved by any particular embodiment.

This disclosure provides, among other things, desensitizing drug products that are useful for the treatment of indications such as premature ejaculation, interstitial cystitis, and vulvodynia, amongst other indications. According to some embodiments, the desensitizing drug formulations may be useful for dermatological conditions and uses to alleviate or lessen the sensation of pain or discomfort. The desensitizing drug product should possess transdermal absorption and efficacy. Preferably, the desensitizing drug product comprises at least one anesthetic agent (or solely one) and additional agents, which, in some embodiments, may include one or more melting point depressing agents. In some embodiments, the desensitizing drug product contains only one anesthetic agent and two melting point depressing agents. In some embodiments, the desensitizing drug product also includes at least one flavoring agent. According to yet another embodiment, the desensitizing drug product includes at least one drug permeation agent.

The terms “about” and “approximately” in the context of numerical values and ranges refers to values or ranges that approximate or are close to the recited values or ranges such that what is being described can perform as intended, such as having a desired rate, amount, degree or extent of absorption and desensitization, as is apparent from the teachings contained herein. Thus, this term encompasses values beyond those simply resulting from systematic error. By “substantially,” as recognized by the skilled person, it is meant in this and similar contexts that the products and methods are suitable for their intended purpose.

All ranges set forth herein in the summary and description and the claims include all numbers or values thereabout or therebetween of the numbers of the range. The ranges expressly denominate and set forth all integers, decimals and fractional values in the range.

The term “treating” in its various grammatical forms refers to, depending on context, avoiding, preventing, curing, reversing, attenuating, alleviating, minimizing, suppressing or halting the deleterious effects of a disease state, disease progression, disease causative agent or other abnormal or undesired condition, as recognized by the skilled person or a person in need of treatment.

The term “premature ejaculation” as used in the present patent application, is a broad term, shall have its ordinary meaning in the industry and is broadly defined and comprises, without limitation, any and all disorders that are characterized by a male ejaculating earlier than he or his partner would want him to during sexual interactions, including sexual intercourse. Premature ejaculation may include situations where a male ejaculates within two minutes of penetration during sexual intercourse. Symptoms of premature ejaculation may include, but are not limited to ejaculation before both sexual partners wish, causing concern or stress, ejaculation that always or nearly always occurs within one or two minutes of penetration during sexual intercourse, the inability to delay ejaculation on all or nearly all penetrations during sexual intercourse, and/or stress or frustration of the avoidance of sexual intimacy as a result of such ejaculation.

The term “interstitial cystitis” as used in the present patent application, is a broad term, shall have its ordinary meaning in the industry and is broadly defined and comprises, without limitation, any and all disorders characterized by a chronic condition characterized by bladder pain. As used herein, methods and compositions for the treatment of interstitial cystitis may also be used to treat or alleviate one or more symptoms of other bladder disorders such as urinary tract infections, overactive bladder, urethritis, urethral syndrome, and prostatitis. Symptoms of interstitial cystitis may include, but are not limited to pain in the pelvis or between the vagina and anus in women or between the scrotum and anus in men, chronic pelvic pain, a persistent, urgent need to urinate, frequent urination, pain during urination, and/or pain during sexual intercourse.

The term “vulvodynia” as used in the present patent application, is a broad term, shall have its ordinary meaning in the industry and is broadly defined, and comprises any and all disorders characterized by a chronic pain syndrome that affects the vulvar area. As used herein, methods and compositions for the treatment of vulvodynia may also be used to treat other female genital pain disorders such as vulvar vestibulitis, characterized by pain only when pressure is applied to the area surrounding the entrance to the vagina. Symptoms of vulvodynia may include, but are not limited to pain in the vulva, including the labia and entrance to the vagina. The pain may be burning, soreness, rawness, throbbing, stinging, irritation, and/or a sharp pain. Symptoms of vulvodynia may also include painful intercourse and itching in the vulvar area. The pain associated with vulvodynia may be constant or intermittent, and may last for months or years.

### Pharmaceutical Compositions Including an Anesthetic Agent

An improved pharmaceutical composition for the treatment of a disorder such as premature ejaculation, interstitial cystitis, or vulvodynia may include at least one anesthetic agent and a first melting point depressing agent.

Anesthetic agents comprise those known in the art and modifications thereof. Such anesthetic agents include lidocaine, tetracaine, procaine, mepivacaine, bupivacaine, etidocaine, and other anesthetic agents. In some embodiments, prilocaine is not present. According to some embodiments, the at least one anesthetic agent may be lidocaine alone. The at least one anesthetic agent can be present in the pharmaceutical composition in an amount in the range of about 0.5% by weight to about 20% by weight, based on the total weight of the pharmaceutical composition. In some embodiments, the at least one anesthetic agent can be present in the pharmaceutical composition in an amount in the range of about 5% by weight to about 10% by weight, based on the total weight of the pharmaceutical composition. In some embodiments, the at least one anesthetic agent can be present in the pharmaceutical composition in an amount in the range of about 6% by weight to about 9% by weight, based on the total weight of the pharmaceutical composition. In some embodiments, the at least one anesthetic agent can be present in the pharmaceutical composition in an amount of about 3% by weight, about 6% by weight, about 8% by weight, or about 9% by weight, based on the total weight of the pharmaceutical composition. For example, in an embodiment, lidocaine can be present in the pharmaceutical composition in an amount of about 7.7% by weight, based on the total weight of the pharmaceutical composition.

Melting point depressing agents also are known in the art, and include thymol, methyl salicylate, phenyl salicylate, butylated hydroxytoluene, butylated hydroxyanisole, S(+)-ibuprofen, R(−)-ibuprofen, cineole, eugenol, capsaicin, eucalyptol, and an alcohol, such as ethyl alcohol, isopropyl alcohol, propylene glycol, polyethylene glycol. Melting point suppressing agents can be used singly or any combination of the above. The presence of one or more melting point depressing agents is believed to allow for a degree of transdermal absorption of the anesthetic agent that is sufficient for the anesthetic agent to desensitize the treated area, such as the penis or the vulvar area. In some embodiments, two different melting point depressing agents are employed. In some embodiments, only one melting point depressing agent is employed. The at least one melting point depressing agent or agents can each be present in the pharmaceutical composition in an amount in the range of about 0.1% by weight to about 20% by weight, based on the total weight of the pharmaceutical composition. In some embodiments, the at least one melting point depressing agent can be present in the pharmaceutical composition in an amount in the range of about 0.5% by weight to about 2% by weight, based on the total weight of the pharmaceutical composition. In some embodiments, the at least one melting point depressing agent can be present in the pharmaceutical composition in an amount in the range of about 6% by weight to about 15% by weight, based on the total weight of the pharmaceutical composition. In some embodiments, the at least one melting point depressing agent can be present in the pharmaceutical composition in an amount of about 0.86% by weight, about 1% by weight, about 5% by weight, or about 10% by weight, based on the total weight of the pharmaceutical composition. In embodiments where two melting point depressing agents are used, a first melting point depressing agent may be present in a desensitizing pharmaceutical formulation in an amount in the range of about 0.5% to about 3% by weight, about 0.5% to about 2.5% by weight, and the like, based on the total weight of the pharmaceutical formulation, and a second melting point depressing agent may be present in the desensitizing pharmaceutical formulation in an amount in the range of about 5% to about 15% by weight, about 8% to about 12% by weight, and the like, based on the total weight of the pharmaceutical formulation. For example, in an embodiment, thymol can be present in the pharmaceutical composition as a first melting point depressing agent in an amount of about 0.86% by weight, based on the total weight of the pharmaceutical composition. In the same or different embodiments, ethanol can be present in the pharmaceutical composition as a second melting point depressing agent in an amount of about 10% by weight, based on the total weight of the pharmaceutical composition. According to some embodiments, an alcohol such as ethanol, if used, is denatured to discourage oral administration of the pharmaceutical composition.

In some embodiments, one or more additional inactive additive ingredients may be added to the formulation in varying amounts to achieve desirable properties in the desensitizing pharmaceutical formulation. The additional inactive additive ingredients may together form the base of the pharmaceutical composition. These inactive additive ingredients may include surfactants, antifoaming agents, flavoring agents, emulsion stabilizers, denaturants, emollients, skin-conditioning agents, fragrance, and/or antioxidants. In some embodiments, acrylates/C10-30 alkyl acrylate crosspolymer such as Carbopol Ultrez 21 may be added to control the consistency and thickness of the formulation in an amount of about 0.20% by weight, based on the total weight of the pharmaceutical composition. In some embodiments, a surfactant, such as cetereareth 20, commercially available as PROCOL CS-20 may be present in the composition in an amount of about 1.25% by weight, based on the total weight of the composition. In some embodiments, an emulsion stabilizer, such as ceterearyl alcohol and/or stearyl alcohol (such as DOW CORNING 580 WAX) may be added to the pharmaceutical composition. If added, the emulsion stabilizer or stabilizers may be present in the composition in an amount in the range of between about 1.0% by weight to about 2.75% by weight, based on the total weight of the composition. In some embodiments, the cetearyl alcohol is present in an amount of about 1.25% by weight and the stearyl alcohol may be present in an amount of about 1.5% by weight. In some embodiments, a chealating agent, such as citric acid, may be present in the composition in an amount of about 0.05% by weight, based on the total weight of the composition. In some embodiments, an anti-foaming agent, such as dimethicone (such as DOW CORNING 200 FLUID 100 CST) may be present in the pharmaceutical composition in an amount of about 1.0% by weight, based on the total weight of the composition. In some embodiments, a fragrance may be added to the composition, such as Harris Coco Verbena WS and/or phenoxyethanol. A fragrance may be present in the pharmaceutical composition in an amount in the range of about 1.0% by weight to about 2.5% by weight, based on the total weight of the pharmaceutical composition. In some embodiments, a fragrance may be Harris Coco Verbena WS and/or phenoxyethanol. According to some embodiments, glycerin may be added to the pharmaceutical formulation. The glycerin may function as a denaturant and to decrease the viscosity of the pharmaceutical formulation. When used, in some embodiments, glycerin may be present in the composition in an amount of about 8.0% by weight, based on the total weight of the composition. In some embodiments, an emollient, such as hydrogenated polyisobutene (available as FANCOL POLYISO-200-CG) and/or macadamia ternifolia seed oil may be added to the pharmaceutical composition. If added, the emollient or emollients may be present in the composition in an amount in the range of between about 3.0% by weight to about 7.0% by weight, based on the total weight of the composition. In some embodiments, the hydrogenated polyisobutene is present in an amount of about 5.0% by weight and the macadamia ternifolia seed oil may be present in an amount of about 1.0% by weight. In some embodiments, an antioxidant such as vitamin E or vitamin E acetate may be present in the composition in an amount of about 0.1% by weight.

According to some embodiments, a flavoring agent may be included in the desensitizing pharmaceutical composition. The flavoring agent may be ethyl vanillin. In some embodiments, ethyl vanillin or another flavoring agent, is present in the pharmaceutical composition in an amount in the range of between about 0.5% by weight to about 5.0% by weight, based on the total weight of the composition. In an embodiment, ethyl vanillin is present in the formulation in amount of about 1.0% by weight of the composition, based on the total weight of the composition. In some embodiments, the 1.0% by weight of the composition of ethyl vanillin replaces 1.0% by weight of the composition of thymol.

According to some embodiments, a desensitizing pharmaceutical composition may be formulated without the use of alcohol, or with only a small amount (up to 1.5% by weight) of alcohol. According to an embodiment, a pharmaceutical composition may comprise lidocaine in an amount of about 10% by weight, ethyl vanillin in an amount of about 1% by weight, sodium bisulfite in an amount of about 0.5% by weight, glycerin in an amount of about 20% by weight, with the remainder of the composition comprising the formulation base. According to some embodiments, the alcohol-free or substantially alcohol free composition comprises glycerin in an amount of about 15% by weight to about 25% by weight, based on the total weight of the alcohol-free or substantially alcohol free composition.

According to some embodiments, a eutectic solution comprising lidocaine, thymol, and ethanol may be created. Examples of eutectic solutions are described in PCT Patent Application Publication No. WO2010/085589, which is hereby incorporated by reference in its entirety.

According to some embodiments, the pharmaceutical compositions may be prepared with various pH buffers. While not meaning to be bound by any theory, it is believed that the varying pH affects the ionization and amount of free base present in the formulation. This may affect the absorption of the formulation into the keratinized and/or non-keratinized skin of a patient. According to some embodiments, the pharmaceutical formulation is prepared with a pH buffered solution of about 7.4, about 7.9, about 8.4, about 8.9, or about 9.9. According to some embodiments, the pH of the buffered solution in the formulation is in the range of about 7.4 to about 8.4.

### Method for Treatment of Premature Ejaculation

According to some embodiments, a desensitizing pharmaceutical composition, as described above, may be applied topically to the penis of a male subject to reduce or eliminate the symptoms of premature ejaculation. Once the desensitizing pharmaceutical composition is applied to the penis, it may be left on the penis for a predetermined amount of time. The desensitizing pharmaceutical composition may then be wiped off of the penis with a cloth, paper towel, or any other suitable wiping means. The desensitizing pharmaceutical composition may be left on the penis for a period of time including between about 5 minutes and about 20 minutes, between about 5 minutes and about 15 minutes, between about 5 minutes and about 10 minutes, and the like.

### Method for Treatment of Interstitial Cystitis

According to some embodiments, a desensitizing pharmaceutical composition, as described above, may be applied topically to the area surrounding the external urethra to reduce or eliminate the symptoms of interstitial cystitis. In some embodiments, the symptom of pain is reduced or eliminated. Once the desensitizing pharmaceutical composition is applied to the urethra, it may be left on the urethra and surrounding area for a predetermined amount of time. The composition may then be wiped off of the urethra and surrounding area with a cloth, paper towel, or any other suitable wiping means. The composition may be left on the urethra and surrounding area for a period of time including between about 5 minutes and about 20 minutes, between about 5 minutes and about 15 minutes, between about 5 minutes and about 10 minutes, and the like. In some embodiments, the composition may be left on the urethra and surrounding area for an extended period of time, such as 2-3 hours, without being wiped off.

### Method for Treatment of Vulvodynia

According to some embodiments, a desensitizing pharmaceutical composition, as described above, may be applied to the vulvar area to reduce or eliminate the symptoms of vulvodynia. In some embodiments, the symptom of pain is reduced or eliminated. Once the desensitizing pharmaceutical composition is applied to the external areas of the vulvar area, which may include the vagina and labia, it may be left on theses areas for a predetermined amount of time. The composition may then be wiped off with a cloth, paper towel, or any other suitable wiping means. The composition may be left on the external areas of the vagina and labia for a period of time including between about 5 minutes and about 20 minutes, between about 5 minutes and about 15 minutes, between about 5 minutes and about 10 minutes, and the like. In some embodiments, the composition may be left on the external areas of the vagina and labia for an extended period of time, such as 2-3 hours, without being wiped off.

### Dermatological Methods of Use

According to some embodiments, a desensitizing pharmaceutical composition, as described in the embodiments above, may be used for its anesthetic effect for various dermatological purposes. For example, the desensitizing pharmaceutical composition may be used to desensitize an area of the skin that is about to be punctured, for example, by a needle for a shot, an IV, or a tattoo.

In some embodiments, a method of use of a desensitizing pharmaceutical composition may include the follow steps. First, the desensitizing pharmaceutical composition may be applied to a general area of skin surrounding and including the skin about to be punctured. Once the desensitizing pharmaceutical composition is applied to the skin, it may be left on the area for a predetermined amount of time. The composition may then be wiped off with a cloth, paper towel, or any other suitable wiping means. The composition may be left on the skin for a period of time including between about 5 minutes and about 20 minutes, between about 5 minutes and about 15 minutes, between about 5 minutes and about 10 minutes, and the like. In some embodiments, the composition may be left on the skin for an extended period of time, such as 2-3 hours, without being wiped off.

Method for Desensitization of Nipple and/or Breast

According to some embodiments, a desensitizing pharmaceutical composition, as described in the embodiments above, may be used for its anesthetic effect on a patient's breast and/or nipple. Various procedures may result in discomfort to the breast and/or nipple of a patient. For example, a breast pap test, such as those available from HALO® may use a device which applies heat, suction, and compression to a patient's nipple and adjacent surrounding breast area to elicit a small amount of fluid from a patient's nipple, which may then be tested for abnormal or precancerous cells. This process may be uncomfortable to the breast and or/nipple of a patient having a breast pap test.

In some embodiments, a method of preventing pain and/or discomfort to the breast and/or nipple of a patient using a desensitizing pharmaceutical composition includes the following steps. First, the desensitizing pharmaceutical composition may be applied to a nipple and/or breast area surrounding the nipple of a patient about to undergo a procedure or test that may be uncomfortable or painful to the breast, such as a breast pap test. Once the desensitizing pharmaceutical composition is applied to the nipple and/or breast area surrounding the nipple to be treated or tested, it may be left on the nipple and/or breast area for a predetermined amount of time. The composition may then be wiped off with a cloth, paper towel, or any other suitable wiping means. The composition may be left on the nipple and/or breast area for a period of time including between about 5 minutes and about 20 minutes, between about 5 minutes and about 15 minutes, between about 5 minutes and about 10 minutes, and the like. The uncomfortable procedure or test may then be performed on the nipple and/or breast area. In some embodiments the patient may experience less pain and/or discomfort after applying and wiping off the desensitizing pharmaceutical composition, then performing the procedure and/or test, than if the patient had not applied and wiped off the desensitizing pharmaceutical composition before performing the same procedure and/or test.

### EXAMPLES

The disclosure is further described by the following non-limiting examples.

### Example 1

Active and inactive ingredients of a desensitizing drug product pharmaceutical composition were prepared, including:
Lidocaine: 7.77%, w/w (weight/weight)
Thymol: 0.86%, w/w
Alcohol SDA 40-B (190 proof): 10%, w/w
Surfactant: 1.25%, w/w
Emulsion Stabilizer: 2.75%, w/w
Chelating Agent: 0.05%, w/w
Antifoaming Agent: 1.0%, w/w
Other additives: 76.32%, w/w

Weights are approximate. The products are formulated for administration as a metered spray.

### Example 2

Active and inactive ingredients of a desensitizing drug product pharmaceutical composition were prepared, including:
Lidocaine: 7.77%, w/w (weight/weight)
Ethyl vanillin: 0.86%, w/w
Ethanol: 10%, w/w
Surfactant: 1.25%, w/w
Emulsion Stabilizer: 2.75%, w/w
Chelating Agent: 0.05%, w/w
Antifoaming Agent: 1.0%, w/w
Other additives: 76.32%, w/w

Weights are approximate. The products are formulated for administration as a metered spray.

### Example 3

The pharmaceutical formulation of EXAMPLE 1 was subjected to flux testing over a range of pH's. The results of such testing are reproduced below. This table illustrates the permeation of lidocaine in the formulation of EXAMPLE 1 through Strat-M (an artificial membrane by Millipore) that replicates human skin permeation and snakeskin, which has been documented to behave like human stratum corneum:

| | Flux (Jₛₛ₎, | Flux (Jₛₛ), |
|---|---|---|
| | μg/cm²/hr | μg/cm²/hr |
| pH | Strat-M | Snakeskin |
| 5.5 | 15 | 17 |
| 6.0 | 24 | na |
| 6.9 | 43 | na |
| 7.4 | 188 | 175 |
| 7.9 | 255 | 251 |
| 8.4 | 251 | 260 |
| 8.9 | 210 | 201 |
| 9.4 | 194 | na |
| 9.9 | 197 | 201 |

Surprisingly, it was found that increased drug permeation occurred at pH between 7.4 and 8.9. This is surprising in view of prior art that teaches that increased drug permeation occurs with the active ingredient is unionized. However, these results show that ionized species of lidocaine (such would exist between pH of 7.4 and 8.9) exhibit improved permeation.

As apparent, the features and attributes of the specific embodiments disclosed herein may be combined in different ways to form additional embodiments, all of which fall within the scope of the present disclosure. In some embodiments, all of these features and embodiments may be implemented based on the systems, methods and devices described herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features or elements. Thus, such conditional language is not generally intended to imply that features or elements are in any way required for one or more embodiments.

It should be emphasized that many variations and modifications may be made to the described embodiments, the elements of which are to be understood as being among other acceptable examples. All such modifications and variations are intended to be included herein within the scope of this disclosure and protected by the following claims.

1. A method of treating interstitial cystitis in a patient, the method comprising:
administering to the patient a pharmaceutical composition comprising 7.7% by weight lidocaine based on the total weight of the pharmaceutical composition, 0.86% thymol based on the total weight of the pharmaceutical composition, 10% by weight ethanol based on the total weight of the pharmaceutical composition, and a base; and
relieving symptoms of interstitial cystitis.

2. The method of claim 1, wherein the pharmaceutical composition does not contain prilocaine.

3. The method of claim 2, wherein the base comprises an aqueous solution, and wherein the aqueous solution comprises a pH in the range of about 7.4 to about 8.9.

4. The method of claim 3, wherein the aqueous solution comprises a pH of about 7.4.

5. The method of claim 3, wherein the aqueous solution comprises a pH of about 7.9.

6. The method of claim 3, wherein the aqueous solution comprises a pH of about 8.4.

7. The method of claim 1, wherein the administering is performed using a metered spray.

8. A method of treating vulvodynia in a patient, the method comprising:
administering to the patient a pharmaceutical composition comprising 7.7% by weight lidocaine based on the total weight of the pharmaceutical composition, 0.86% thymol based on the total weight of the pharmaceutical composition, 10% by weight ethanol based on the total weight of the pharmaceutical composition, and a base; and
relieving symptoms of vulvodynia.

9. The method of claim 8, wherein the pharmaceutical composition does not contain prilocaine.

10. The method of claim 9, wherein the base comprises an aqueous solution, and wherein the aqueous solution comprises a pH in the range of about 7.4 to about 8.9.

11. The method of claim 10, wherein the aqueous solution comprises a pH of about 7.4.

12. The method of claim 10, wherein the aqueous solution comprises a pH of about 7.9.

13. The method of claim 10, wherein the aqueous solution comprises a pH of about 8.4.

14. The method of claim 9, wherein the administering is performed using a metered spray.

15. A method of preventing pain to the breast of a patient, the method comprising:
administering to the patient a pharmaceutical composition comprising 7.7% by weight lidocaine based on the total weight of the pharmaceutical composition, 0.86% thymol based on the total weight of the pharmaceutical composition, 10% by weight ethanol based on the total weight of the pharmaceutical composition, and a base; and
preventing pain to the breast of a patient.

16. The method of claim 15, wherein the pharmaceutical composition does not contain prilocaine.

17. The method of claim 16, wherein the base comprises an aqueous solution, and wherein the aqueous solution comprises a pH in the range of about 7.4 to about 8.9.

18. The method of claim 16, wherein the administering is performed using a metered spray.
